(19) United States
(12) Patent Application Publication
(10) Pub. No.: US 2011/0015215 A1
(43) Pub. Date: Jan. 20, 2011

(54) CRYSTALLINE FORMS OF 4-[6-(6-METHANESULFONYL-2-METHYL-PYRIDIN-3-YLAMINO)-5-METHOXY-PYRIMIDIN-4-YLOXY]-PIPERIDINE-1-CARBOXYLIC ACID ISOPROPYL ESTER

(76) Inventors: Paul Deghetto, Bound Brook, NJ (US); Nagy E. Fawzy, Piscataway, NJ (US); Gary Fech, Perth Amboy, NJ (US); Kenneth M. Wells, Hillsborough, NJ (US); Wenju Wu, Modesto, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/783,913

(22) Filed: May 20, 2010

Related U.S. Application Data
(60) Provisional application No. 61/179,779, filed on May 20, 2009.

(51) Int. Cl. A61K 31/506; C07D 401/14; A61P 3/10; A61P 3/00; A61P 3/06; A61P 3/04

(52) U.S. Cl. 514/269; 544/319

(57) ABSTRACT

The present invention is directed to a novel crystalline forms of 4-[6-(6-methanesulfonyl-2-methyl-pyridin-3-ylamino)-5-methoxy-pyrimidin-4-yloxy]-piperidine-1-carboxylic acid isopropyl ester, pharmaceutical compositions containing said crystalline form and the use of said crystalline forms in the treatment of metabolic related disorders. The present invention is further directed to processes for the preparation of the crystalline forms of 4-[6-(6-methanesulfonyl-2-methyl-pyridin-3-ylamino)-5-methoxy-pyrimidin-4-yloxy]-piperidine-1-carboxylic acid isopropyl ester.

### CROSS REFERENCE TO RELATED APPLICATIONS

This application claims the benefit of U.S. Provisional Application 61/179,779, filed on May 20, 2009, which is incorporated by reference herein in its entirety.

### FIELD OF THE INVENTION

The present invention is directed to novel crystalline forms of the GPR119 agonist 4-[6-(6-Methanesulfonyl-2-methyl-pyridin-3-ylamino)-5-methoxy-pyrimidin-4-yloxy]-piperidine-1-carboxylic acid isopropyl ester, pharmaceutical compositions containing said crystalline forms and the use of said crystalline forms in the treatment of metabolic related disorders. The present invention is further directed to processes for the preparation of the crystalline forms of 4-[6-(6-Methanesulfonyl-2-methyl-pyridin-3-ylamino)-5-methoxy-pyrimidin-4-yloxy]-piperidine-1-carboxylic acid isopropyl ester.

### BACKGROUND OF THE INVENTION

Diabetes mellitus is a serious disease afflicting over 100 million people worldwide. In the United States, there are more than 12 million diabetics, with 600,000 new cases diagnosed each year.

Diabetes mellitus is a diagnostic term for a group of disorders characterized by abnormal glucose homeostasis resulting in elevated blood sugar. There are many types of diabetes, but the two most common are Type I (also referred to as insulin-dependent diabetes mellitus or IDDM) and Type II (also referred to as non-insulin-dependent diabetes mellitus or NIDDM).

The etiology of the different types of diabetes is not the same; however, everyone with diabetes has two things in common: overproduction of glucose by the liver and little or no ability to move glucose out of the blood into the cells where it becomes the body's primary fuel.

People who do not have diabetes rely on insulin, a hormone made in the pancreas, to move glucose from the blood into the cells of the body. However, people who have diabetes either don't produce insulin or can't efficiently use the insulin they produce; therefore, they can't move glucose into their cells. Glucose accumulates in the blood creating a condition called hyperglycemia, and over time, can cause serious health problems.

Diabetes is a syndrome with interrelated metabolic, vascular, and neuropathic components. The metabolic syndrome, generally characterized by hyperglycemia, comprises alterations in carbohydrate, fat and protein metabolism caused by absent or markedly reduced insulin secretion and/or ineffective insulin action. The vascular syndrome consists of abnormalities in the blood vessels leading to cardiovascular, retinal and renal complications. Abnormalities in the peripheral and autonomic nervous systems are also part of the diabetic syndrome.

People with IDDM, which accounts for about 5% to 10% of those who have diabetes, don't produce insulin and therefore must inject insulin to keep their blood glucose levels normal. IDDM is characterized by low or undetectable levels of endogenous insulin production caused by destruction of the insulin-producing β cells of the pancreas, the characteristic that most readily distinguishes IDDM from NIDDM. IDDM, once termed juvenile-onset diabetes, strikes young and older adults alike.

Approximately 90 to 95% of people with diabetes have Type II (or NIDDM). NIDDM subjects produce insulin, but the cells in their bodies are insulin resistant: the cells don't respond properly to the hormone, so glucose accumulates in their blood. NIDDM is characterized by a relative disparity between endogenous insulin production and insulin requirements, leading to elevated blood glucose levels. In contrast to IDDM, there is always some endogenous insulin production in NIDDM; many NIDDM patients have normal or even elevated blood insulin levels, while other NIDDM patients have inadequate insulin production (ROTWEIN, R. et al., “Polymorphism in the 5′ flanking region of the human insulin gene: a genetic marker for non-insulin dependent diabetes”, N. Engl. J. Med., 1983, pp 65-71, Vol. 308). Most people diagnosed with NIDDM are age 30 or older, and half of all new cases are age 55 and older. Compared with whites and Asians, NIDDM is more common among Native Americans, African-Americans, Latinos, and Hispanics. In addition, the onset can be insidious or even clinically inapparent, making diagnosis difficult.

The primary pathogenic lesion on NIDDM has remained elusive. Many have suggested that primary insulin resistance of the peripheral tissues is the initial event. Genetic epidemiological studies have supported this view. Similarly, insulin secretion abnormalities have been argued as the primary defect in NIDDM. It is likely that both phenomena are important contributors to the disease process (RIMOIN, D. L., et. al., Emery and Rimoin's Principles and Practice of Medical Genetics 3^{rd} Ed., 1996, pp 1401-1402, Volume 1).

Many people with NIDDM have sedentary lifestyles and are obese; they weigh approximately 20% more than the recommended weight for their height and build. Furthermore, obesity is characterized by hyperinsulinemia and insulin resistance, a feature shared with NIDDM, hypertension and atherosclerosis.

Obesity and diabetes are among the most common human health problems in industrialized societies. In industrialized countries a third of the population is at least 20% overweight. In the United States, the percentage of obese people has increased from 25% at the end of the 1970s, to 33% at the beginning the 1990s. Obesity is one of the most important risk factors for NIDDM. Definitions of obesity differ, but in general, a subject weighing at least 20% more than the recommended weight for his/her height and build is considered obese. The risk of developing NIDDM is tripled in subjects 30% overweight, and three-quarters with NIDDM are overweight.

Obesity, which is the result of an imbalance between caloric intake and energy expenditure, is highly correlated with insulin resistance and diabetes in experimental animals and human. However, the molecular mechanisms that are involved in obesity-diabetes syndromes are not clear. During early development of obesity, increase insulin secretion balances insulin resistance and protects patients from hyperglycemia (LE STUNFF, C, et al., “Early Changes in Postprandial Insulin Secretion, Not in Insulin Sensitivity, Characterize Juvenile Obesity”, Diabetes, 1994, pp 696-702, Vol. 43). However, after several decades, β cell function deteriorates and non-insulin-dependent diabetes develops in about 20% of the obese population (PEDERSON, P., “The Impact of Obesity on the Pathogenesis of Non-Insulin-Dependent Diabetes Mellitus: A Review of Current Hypotheses”, Diab. Metab. Rev., 1989, pp 505-509, Vol. 5) and (BRANCATI, F. L., et al., “Body Weight Patterns From 20 to 49 Years of Age and Subsequent Risk for Diabetes Mellitus: The Johns Hopkins Precursors Study”, Arch. Intern. Med., 1999, pp 957-963, Vol. 159). Given its high prevalence in modern societies, obesity has thus become the leading risk factor for NIDDM (HILL, J. O., et al., “Environmental contributions to the obesity epidemic”, Science, 1998, pp 1371-1374, Vol. 280). However, the factors which predispose a fraction of patients to alteration of insulin secretion in response to fat accumulation remain unknown.

Whether someone is classified as overweight or obese is generally determined on the basis of their body mass index (BMI) which is calculated by dividing body weight (kg) by height squared (m²). Thus, the units of BMI are kg/m² and it is possible to calculate the BMI range associated with minimum mortality in each decade of life. Overweight is defined as a BMI in the range 25-30 kg/m², and obesity as a BMI greater than 30 kg/m² (see TABLE below). There are problems with this definition in that it does not take into account the proportion of body mass that is muscle in relation to fat (adipose tissue). To account for this, obesity can also be defined on the basis of body fat content: greater than 25% and 30% in males and females, respectively.

CLASSIFICATION OF WEIGHT
BY BODY MASS INDEX (BMI)

| BMI | CLASSIFICATION |
|---|---|
| <18.5 | Underweight |
| 18.5-24.9 | Normal |
| 25.0-29.9 | Overweight |
| 30.0-34.9 | Obesity (Class I) |
| 35.0-39.9 | Obesity (Class II) |
| >40 | Extreme Obesity (Class III) |

As the BMI increases there is an increased risk of death from a variety of causes that is independent of other risk factors. The most common diseases with obesity are cardiovascular disease (particularly hypertension), diabetes (obesity aggravates the development of diabetes), gall bladder disease (particularly cancer) and diseases of reproduction. Research has shown that even a modest reduction in body weight can correspond to a significant reduction in the risk of developing coronary heart disease.

Compounds marketed as anti-obesity agents include Orlistat (XENICAL™) and Sibutramine. Orlistat (a lipase inhibitor) inhibits fat absorption directly and tends to produce a high incidence of unpleasant (though relatively harmless) side-effects such as diarrhea. Sibutramine (a mixed 5-HT/noradrenaline reuptake inhibitor) can increase blood pressure and heart rate in some patients. The serotonin releaser/reuptake inhibitors fenfluramine (Pondimin™) and dexfenfluramine (Redux™) have been reported to decrease food intake and body weight over a prolonged period (greater than 6 months). However, both products were withdrawn after reports of preliminary evidence of heart valve abnormalities associated with their use. Accordingly, there is a need for the development of a safer anti-obesity agent.

Obesity considerably increases the risk of developing cardiovascular diseases as well. Coronary insufficiency, atheromatous disease, and cardiac insufficiency are at the forefront of the cardiovascular complication induced by obesity. It is estimated that if the entire population had an ideal weight, the risk of coronary insufficiency would decrease by 25% and the risk of cardiac insufficiency and of cerebral vascular accidents by 35%. The incidence of coronary diseases is doubled in subjects less than 50 years of age who are 30% overweight. The diabetes patient faces a 30% reduced lifespan. After age 45, people with diabetes are about three times more likely than people without diabetes to have significant heart disease and up to five times more likely to have a stroke. These findings emphasize the inter-relations between risks factors for NIDDM and coronary heart disease and the potential value of an integrated approach to the prevention of these conditions based on the prevention of these conditions based on the prevention of obesity (PERRY, I. J., et al., “Prospective study of risk factors for development of non-insulin dependent diabetes in middle aged British men”, British Med J., 1995, pp 560-564, Vol. 310).

Diabetes has also been implicated in the development of kidney disease, eye diseases and nervous-system problems. Kidney disease, also called nephropathy, occurs when the kidney's “filter mechanism” is damaged and protein leaks into urine in excessive amounts and eventually the kidney fails. Diabetes is also a leading cause of damage to the retina at the back of the eye and increases risk of cataracts and glaucoma. Finally, diabetes is associated with nerve damage, especially in the legs and feet, which interferes with the ability to sense pain and contributes to serious infections. Taken together, diabetes complications are one of the nation's leading causes of death.

### SUMMARY OF THE INVENTION

The present invention is directed to novel crystalline forms of the compound of formula (A)

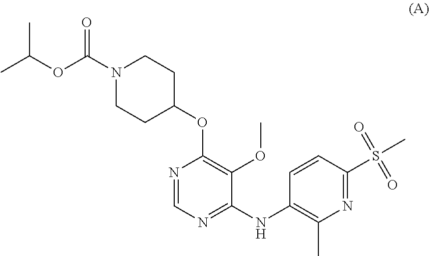

which compound is also known as 4-[6-(6-Methanesulfonyl-2-methyl-pyridin-3-ylamino)-5-methoxy-pyrimidin-4-yloxy]-piperidine-1-carboxylic acid isopropyl ester. Each of the novel crystalline forms of the compound of formula (A), hereinafter referred to as crystalline form (A-I), crystalline form (A-IV) and crystalline form (A-VI), may be characterized, for example, by its powder X-ray diffraction (i.e., pXRD) pattern.

Illustrative of the invention are pharmaceutical compositions comprising a pharmaceutically acceptable carrier and any of the crystalline forms of the compound of formula (A) as herein described. An illustration of the invention is a pharmaceutical composition made by mixing any of the crystalline forms of the compound of formula (A) as herein described and a pharmaceutically acceptable carrier. Illustrating the invention is a process for making a pharmaceutical composition comprising mixing any of the crystalline forms of the compound of formula (A) as herein described and a pharmaceutically acceptable carrier.

Exemplifying the invention are methods of treating a metabolic related disorder (selected from the group consisting of hyperlipidemia, type 1 diabetes, type 2 diabetes mellitus, idiopathic type 1 diabetes (Type 1b), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance) comprising administering to a subject in need thereof a therapeutically effective amount of any of the crystalline forms of the compound of formula (A) as herein described.

The present invention is further directed to any of the crystalline forms of the compound of formula (A) as herein described for use in a method of treatment of the human or animal body by therapy.

The present invention is further directed to any of the crystalline forms of the compound of formula (A) as herein described for use in a method of treatment of a metabolic related disorder.

The present invention is further directed to any of the crystalline forms of the compound of formula (A) as herein described for use in a method of treatment of a metabolic related disorder selected from the group consisting of Type I diabetes, Type II diabetes, inadequate glucose tolerance, insulin resistance, hyperglycemia, hyperlipidemia, hypertriglyceridemia, hypercholesterolemia, dyslipidemia, and Syndrome X.

The present invention is further directed to any of the crystalline forms of the compound of formula (A) as herein described for treatment of Type II diabetes.

The present invention is further directed to any of the crystalline forms of the compound of formula (A) as herein described for use in a method of (a) decreasing food intake, (b) inducing satiety, (c) controlling weight gain, or (d) decreasing weight gain, in a subject in need thereof.

The present invention is further directed to methods for the treatment of metabolic related disorders (including, but not limited to, Type I diabetes, Type II diabetes, inadequate glucose tolerance, insulin resistance, hyperglycemia, hyperlipidemia, hypertriglyceridemia, hypercholesterolemia, dyslipidemia or Syndrome X), comprising administering to a subject in need thereof a therapeutically effective amount of any of the crystalline forms of the compound of formula (A) as herein described.

The present invention is further directed to methods for decreasing food intake, inducing satiety, controlling weight gain or decreasing weight gain, comprising administering to a subject in need thereof a therapeutically effective amount of any of the crystalline forms of the compound of formula (A) as herein described.

The present invention is further directed to the use of any of the crystalline forms as described herein for the preparation of a medicament for treating a metabolic disorder in a subject in need thereof.

The present invention is further directed to the use of any of the crystalline forms of the compound of formula (A) as herein described for the preparation of a medicament for treating: (a) Type I diabetes, (b) Type II diabetes, (c) inadequate glucose tolerance, (d) insulin resistance, (e) hyperglycemia, (f) hyperlipidemia, (g) hypertriglyceridemia, (h) hypercholesterolemia, (i) dyslipidemia, or (j) Syndrome X, in a subject in need thereof.

The present invention is further directed to the use of any of the crystalline forms as described herein for the preparation of a medicament for treating Type II diabetes in a subject in need thereof.

The present invention is further directed to the use of any of the crystalline forms of the compound of formula (A) as herein described for the preparation of a medicament for (a) decreasing food intake, (b) inducing satiety, (c) controlling weight gain or (d) decreasing weight gain, in a subject in need thereof.

### BRIEF DESCRIPTION OF THE FIGURES

FIG. 1 illustrates a representative powder XRD pattern of crystalline form (A-I), measured as described herein.

FIG. 2 illustrates a representative DSC for crystalline form (A-I), measured as described herein.

FIG. 3 illustrates a representative TGA scan for crystalline form (A-I), measured as described herein.

FIG. 4 illustrates a representative powder XRD pattern of crystalline form (A-IV), measured as described herein.

FIG. 5 illustrates a representative DSC for crystalline form (A-IV), measured as described herein.

FIG. 6 illustrates a representative TGA scan for crystalline form (A-IV), measured as described herein.

FIG. 7 illustrates a representative powder XRD pattern of crystalline form (A-VI), measured as described herein.

FIG. 8 illustrates a representative DSC for crystalline form (A-VI), measured as described herein.

FIG. 9 illustrates a representative TGA scan for crystalline form (A-VI), measured as described herein.

### DETAILED DESCRIPTION OF THE INVENTION

The present invention is directed to novel crystalline forms of the compound of formula (A)

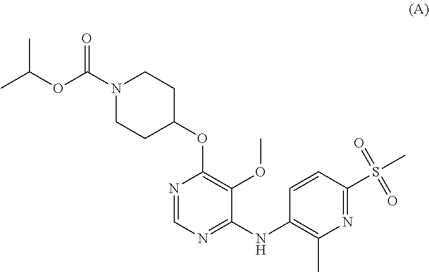

hereinafter referred to as crystalline form (A-I), crystalline form (A-IV) and crystalline form (A-VI). Crystalline form (A-I) of the compound of formula (A) is anhydrous (i.e. a non-hydrate form), with a peak melting temperature (as measured by DSC) of about 166.8° C. Crystalline form (A-IV) of the compound of formula (A) of the present invention is anhydrous (i.e. a non-hydrate form), with a peak melting temperature (as measured by DSC) of about 155.6° C. Crystalline form (A-VI) of the compound of formula (A) of the present invention is an anhydrous form, with a peak melting temperature (as measured by DSC) of about 164.0° C.

The present invention is further directed to a process for the preparation of the novel crystalline forms of the compound of formula (A) as herein described. The present invention is further directed to pharmaceutical compositions comprising any of the crystalline forms of the compound of formula (A) as described herein. The present invention is further directed to the use of any of the crystalline forms of the compound of formula (A) as described herein for the treatment of metabolic related disorders.

Jones, R. M., et al., in US Patent Publication 2007/0167473 A1, published Jul. 19, 2007, which is incorporated by reference in its entirety herein, disclose the compound of formula (A), methods for the preparation of the compound of formula (A) and methods of treatment using the compound of formula (A). The compound of formula (A) is a selective GDIR (glucose-dependent insulin receptor) agonist useful for the treatment of glucose related disorders, including, but not limited to, Type I diabetes, Type II diabetes, inadequate glucose tolerance, insulin resistance, hyperglycemia, hyperlipidemia, hypertriglyceridemia, hypercholesterolemia, dyslipidemia, or Syndrome X.

In an embodiment, the present invention is directed to methods for the treatment of a metabolic related disorder comprising administering to a subject in need thereof a therapeutically effective amount of any of the crystalline form of the compound of formula (A) as described herein.

In some embodiments of the present invention, the metabolic-related disorder is selected from the group consisting of hyperlipidemia, type 1 diabetes, type 2 diabetes mellitus, idiopathic type 1 diabetes (Type 1b), latent autoimmune diabetes in adults (LADA), early-onset type 2 diabetes (EOD), youth-onset atypical diabetes (YOAD), maturity onset diabetes of the young (MODY), malnutrition-related diabetes, gestational diabetes, coronary heart disease, ischemic stroke, restenosis after angioplasty, peripheral vascular disease, intermittent claudication, myocardial infarction (e.g. necrosis and apoptosis), dyslipidemia, post-prandial lipemia, conditions of impaired glucose tolerance (IGT), conditions of impaired fasting plasma glucose, metabolic acidosis, ketosis, arthritis, obesity, osteoporosis, hypertension, congestive heart failure, left ventricular hypertrophy, peripheral arterial disease, diabetic retinopathy, macular degeneration, cataract, diabetic nephropathy, glomerulosclerosis, chronic renal failure, diabetic neuropathy, metabolic syndrome, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertrygliceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction, and impaired vascular compliance.

In another embodiment of the present invention, the metabolic related disorder is selected from the group consisting of Type I diabetes, Type II diabetes, inadequate glucose tolerance, insulin resistance, hyperglycemia, hyperlipidemia, hypertriglyceridemia, hypercholesterolemia, dyslipidemia, and Syndrome X. In an embodiment of the preset invention, the metabolic-related disorder is selected from the group consisting of Type I diabetes, Type II diabetes, inadequate glucose tolerance, insulin resistance, hyperglycemia, hyperlipidemia, hypertriglyceridemia, hypercholesterolemia, dyslipidemia, and Syndrome X. In another embodiment of the present invention, the metabolic-related disorder is Type II diabetes. In another embodiment of the present invention, the metabolic-related disorder is hyperglycemia. In another embodiment of the present invention, the metabolic-related disorder is hyperlipidemia. In another embodiment of the present invention, the metabolic-related disorder is hypertriglyceridemia. In another embodiment of the present invention, the metabolic-related disorder is Type I diabetes. In another embodiment of the present invention, the metabolic-related disorder is dyslipidemia. In another embodiment of the present invention, the metabolic-related disorder is Syndrome X.

The present invention is further directed to methods for decreasing food intake, inducing satiety, controlling weight gain or decreasing weight gain, comprising administering to a subject in need thereof a therapeutically effective amount of any of the crystalline form of the compound of formula (A) as described herein. In another embodiment, the present invention is directed to the use of any of the crystalline forms of the compound of formula (A) as described herein for the treatment of a metabolic-related disorder, wherein the metabolic related disorder is obesity.

In some embodiments, the present invention is directed to method of treating human patients whose body mass index is in the range of from about 18.5 to about 45. In some embodiments, the human has a body mass index of from about 25 to about 45. In some embodiments, the human has a body mass index of from about 30 to about 45. In some embodiments, the human has a body mass index of from about 35 to about 45.

In addition to the foregoing beneficial uses, the crystalline forms of the compound of formula (A) are further useful in the treatment of additional diseases, including, without limitation, the following.

The most significant pathologies in Type II diabetes are impaired insulin signaling at its target tissues (“insulin resistance”) and failure of the insulin-producing cells of the pancreas to secrete an appropriate degree of insulin in response to a hyperglycemic signal. Current therapies to treat the latter include inhibitors of the β-cell ATP-sensitive potassium channel to trigger the release of endogenous insulin stores, or administration of exogenous insulin. Neither of these achieves accurate normalization of blood glucose levels and both carry the risk of inducing hypoglycemia. For these reasons, there has been intense interest in the development of pharmaceuticals that function in a glucose-dependent action, i.e. potentiators of glucose signaling. Physiological signaling systems which function in this manner are well-characterized and include the gut peptides GLP1, GIP and PACAP. These hormones act via their cognate G-protein coupled receptor to stimulate the production of cAMP in pancreatic β-cells. The increased cAMP does not appear to result in stimulation of insulin release during the fasting or preprandial state. However, a series of biochemical targets of cAMP signaling, including the ATP-sensitive potassium channel, voltage-sensitive potassium channels and the exocytotic machinery, are modified in such a way that the insulin secretory response to a postprandial glucose stimulus is markedly enhanced. Accordingly, agonists of novel, similarly functioning, β-cell GPCRs, including GPR119, would also stimulate the release of endogenous insulin and consequently promote normoglycemia in Type II diabetes.

It is also established that increased cAMP, for example as a result of GLP1 stimulation, promotes β-cell proliferation, inhibits β-cell death and thus improves islet mass. This positive effect on β-cell mass is expected to be beneficial in both Type II diabetes, where insufficient insulin is produced, and Type I diabetes, where β-cells are destroyed by an inappropriate autoimmune response.

Some β-cell GPCRs, including GPR119, are also present in the hypothalamus where they modulate hunger, satiety, decrease food intake, controlling or decreasing weight and energy expenditure. Hence, given their function within the hypothalamic circuitry, agonists or inverse agonists of these receptors mitigate hunger, promote satiety and therefore modulate weight.

It is also well-established that metabolic diseases exert a negative influence on other physiological systems. Thus, there is often the co-development of multiple disease states (e.g. type I diabetes, type II diabetes, inadequate glucose tolerance, insulin resistance, hyperglycemia, hyperlipidemia, hypertriglyceridemia, hypercholesterolemia, dyslipidemia, obesity or cardiovascular disease in “Syndrome X”) or secondary diseases which clearly occur secondary to diabetes (e.g. kidney disease, peripheral neuropathy). Thus, it is expected that effective treatment of the diabetic condition will in turn be of benefit to such interconnected disease states.

One aspect of the present invention pertains to methods of modulating a GPR119 receptor in an individual comprising contacting the receptor with any of the crystalline form of the compound of formula (A) as described herein. In some embodiments, the modulation of the GPR119 receptor is treatment of a metabolic-related disorder and complications thereof. In some embodiments, the metabolic-related disorder is type I diabetes, type II diabetes, inadequate glucose tolerance, insulin resistance, hyperglycemia, hyperlipidemia, hypertriglyceridemia, hypercholesterolemia, dyslipidemia or syndrome X. In some embodiments, the metabolic-related disorder is type II diabetes. In some embodiments, the metabolic-related disorder is hyperglycemia. In some embodiments, the metabolic-related disorder is hyperlipidemia. In some embodiments, the metabolic-related disorder is hypertriglyceridemia. In some embodiments, the metabolic-related disorder is type I diabetes. In some embodiments, the metabolic-related disorder is dyslipidemia. In some embodiments, the metabolic-related disorder is syndrome X. In some embodiments, the individual is a mammal. In some embodiments, the mammal is a human.

Some embodiments of the present invention include a method of modulating a GPR119 receptor in an individual comprising contacting the receptor with any of the crystalline form of the compound of formula (A) as described herein, wherein the modulation of the GPR119 receptor reduces food intake of the individual. In some embodiments the individual is a mammal. In some embodiments the mammal is a human. In some embodiments the human has a body mass index of about 18.5 to about 45. In some embodiments the human has a body mass index of about 25 to about 45. In some embodiments the human has a body mass index of about 30 to about 45. In some embodiments the human has a body mass index of about 35 to about 45.

Some embodiments of the present invention include a method of modulating a GPR119 receptor in an individual comprising contacting the receptor with any of the crystalline form of the compound of formula (A) as described herein, wherein the modulation of the GPR119 receptor induces satiety in the individual. In some embodiments the individual is a mammal. In some embodiments the mammal is a human. In some embodiments the human has a body mass index of about 18.5 to about 45. In some embodiments the human has a body mass index of about 25 to about 45. In some embodiments the human has a body mass index of about 30 to about 45. In some embodiments the human has a body mass index of about 35 to about 45.

Some embodiments of the present invention include a method of modulating a GPR119 receptor in an individual comprising contacting the receptor with any of the crystalline form of the compound of formula (A) as described herein, wherein the modulation of the GPR119 receptor controls or reduces weight gain of the individual. In some embodiments the individual is a mammal. In some embodiments the mammal is a human. In some embodiments the human has a body mass index of about 18.5 to about 45. In some embodiments the human has a body mass index of about 25 to about 45. In some embodiments the human has a body mass index of about 30 to about 45. In some embodiments the human has a body mass index of about 35 to about 45.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As used herein, unless otherwise noted, the term “isolated form” shall mean that the compound is present in a form which is separate from any solid mixture with another compound(s), solvent system or biological environment. In an embodiment of the present invention, crystalline form (A-I) of the compound of formula (A) is present as an isolated form. In another embodiment of the present invention, crystalline form (A-IV) of the compound of formula (A) is present as an isolated form. In another embodiment of the present invention, crystalline form (A-VI) of the compound of formula (A) is present as an isolated form.

As used herein, unless otherwise noted, the term “substantially pure form” shall mean that the mole percent of impurities in the isolated compound or crystalline form is less than about 5 mole percent, preferably less than about 2 mole percent, more preferably, less than about 0.5 mole percent, most preferably, less than about 0.1 mole percent. In an embodiment of the present invention, crystalline form (A-I) of the compound of formula (A) is present as a substantially pure form. In another embodiment of the present invention, crystalline form (A-IV) of the compound of formula (A) is present as a substantially pure form. In another embodiment of the present invention, crystalline form (A-VI) of the compound of formula (A) is present as a substantially pure form.

As used herein, unless otherwise noted, the term “substantially free of other polymorph or crystalline form(s)” when used to described a crystalline form of the compound of formula (A) shall mean that mole percent of other polymorph or crystalline form(s) in the isolated crystalline form is less than about 5 mole percent, preferably less than about 2 mole percent, more preferably, less than about 0.5 mole percent, most preferably less than about 0.1 mole percent. In an embodiment of the present invention, crystalline form (A-I) of the compound of formula (A) is present as a form substantially free of other polymorph or crystalline form(s). In another embodiment of the present invention, crystalline form (A-IV) of the compound of formula (A) is present as a form substantially free of other polymorph or crystalline form(s). In another embodiment of the present invention, crystalline form (A-VI) of the compound of formula (A) is present as a form substantially free of other polymorph or crystalline form(s).

One skilled in the art will recognize that, where not otherwise specified, the reaction step(s) is performed under suitable conditions, according to known methods, to provide the desired product.

One skilled in the art will recognize that wherein a reaction step of the present invention may be carried out in a variety of solvents or solvent systems, said reaction step may also be carried out in a mixture of the suitable solvents or solvent systems. One skilled in the art will recognize that, in the specification and claims as presented herein, wherein a reagent or reagent class/type (e.g. base, solvent, etc.) is recited in more than one step of a process, the individual reagents are independently selected for each reaction step and may be the same of different from each other. For example wherein two steps of a process recite an organic or inorganic base as a reagent, the organic or inorganic base selected for the first step may be the same or different than the organic or inorganic base of the second step.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any range therein.

Crystalline forms (A-I), (A-IV) and (A-VI) may be prepared according to the processes as described in Example 1-4, which follow hereinafter.

Powder X-Ray Diffraction (pXRD)

The crystalline forms of the compound of formula (A) were characterized as to their powder X-ray diffraction patterns (pXRD), for example as follows. The sample was examined using an x-ray diffractometer (Bruker AXS Model D8 Advance) equipped with Gobel mirror incident beam and PSD detector (type lynxEye). The sample was placed on to zero-background holder and scanned under ambient conditions of temperature and humidity. The sample was scanned from 3 to 40°2θ at a step size of 0.019°2θ and a time per step of 38.4 seconds. The radiation was CuKα (45 KkV and 40 mA). The divergence slit and anti-scatter slit were 0.982° and 0.499°, respectively.

One skilled in the art will recognize that the pXRD measured values which follow herein (°2Theta, FWHM, d-spacing and % Relative Intensity) will vary with various parameters including, but not limited to, precision and method of grinding during sample preparation, crystal size and morphology, diffractometer configuration, and data collection parameters/experimental conditions. One skilled in the art will further recognize that the crystal forms of the present invention are not limited to crystalline forms which provide a powder X-ray diffraction pattern, and/or peak characteristics identical to those described in the Tables and Figures which follow herein. Notwithstanding, one skilled in the art will recognize that any crystalline forms of the compound of formula (A) which provide a powder x-ray diffraction pattern and/or peak characteristics which are substantially similar to those described in the Tables and Figures which follow herein, shall fall within the scope of this invention.

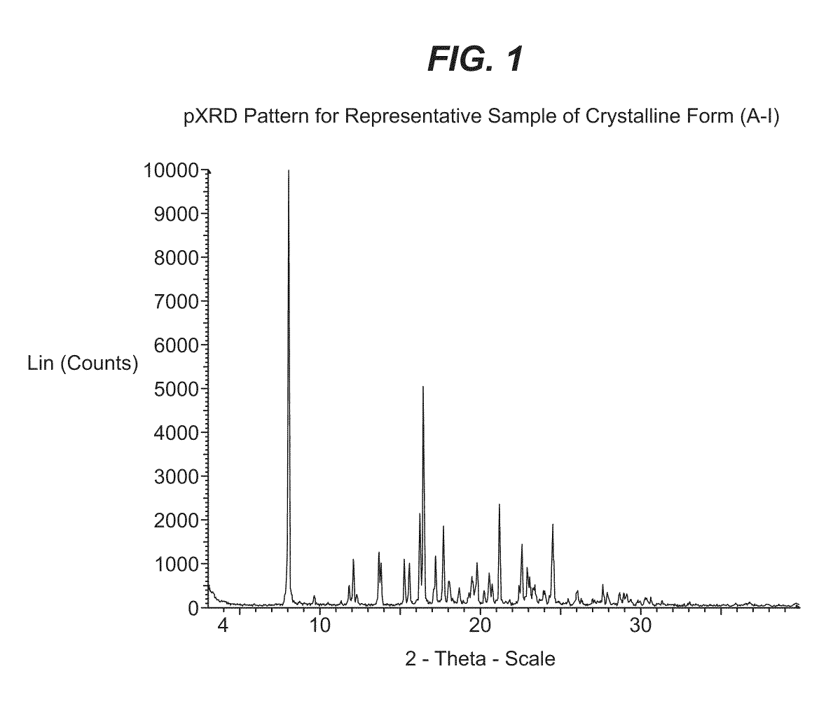

The powder XRD spectrum was measured for a representative sample of crystalline form (A-I) of the compound of formula (A), as shown in FIG. 1. Crystalline form (A-I) of the compound of formula (A) may be characterized by its powder X-ray diffraction pattern, which comprised the peaks as listed in Table 1, below.

TABLE 1
pXRD Peaks, Crystalline Form (A-I)

| Pos. [°2Th.] | d-spacing [Å] | Rel. Int. [%] |
|---|---|---|
| 8.0 | 11.0 | 100 |
| 12.1 | 7.3 | 10 |
| 13.6 | 6.5 | 12 |
| 13.8 | 6.4 | 10 |
| 15.2 | 5.8 | 10 |
| 15.6 | 5.7 | 9 |
| 16.2 | 5.5 | 20 |
| 16.4 | 5.4 | 51 |
| 17.2 | 5.1 | 11 |
| 17.7 | 5.0 | 18 |
| 19.8 | 4.5 | 10 |
| 21.2 | 4.2 | 23 |
| 22.6 | 3.9 | 14 |
| 22.9 | 3.9 | 9 |
| 24.5 | 3.6 | 18 |

Preferably, crystalline form (A-I) of the compound of formula (A) is characterized by its pXRD pattern which comprises peaks having a relative intensity greater than or equal to about 10%, more preferably, greater than or equal to about 15%, as listed in Table 2, below; more preferably, greater than or equal to about 20%.

TABLE 2
pXRD Peaks, Crystalline Form (A-I)

| Pos. [°2Th.] | d-spacing [Å] | Rel. Int. [%] |
|---|---|---|
| 8.0 | 11.0 | 100 |
| 16.2 | 5.5 | 20 |
| 16.4 | 5.4 | 51 |
| 17.7 | 5.0 | 18 |
| 21.2 | 4.2 | 23 |
| 24.5 | 3.6 | 18 |

In an embodiment, crystalline form (A-I) of the compound of formula (A) is characterized by its pXRD pattern which comprises the peaks listed in Table 3, below.

TABLE 3
pXRD Peaks, Crystalline Form (A-I)

| Pos. [°2Th.] | d-spacing [Å] |
|---|---|
| 8.0 | 11.0 |
| 16.4 | 5.4 |
| 17.7 | 5.0 |
| 21.2 | 4.2 |
| 24.5 | 3.6 |

In one embodiment, crystalline form (A-I) of the compound of formula (A) has an X-ray powder diffraction pattern comprising a peak, in terms of 2θ, at about 8.0°. In another embodiment, crystalline form (A-I) of the compound of formula (A) has an X-ray powder diffraction pattern comprising a peak, in terms of 2θ, at about 8.0° and about 17.8°. In yet another embodiment, crystalline form (A-I) of the compound of formula (A) has an X-ray powder diffraction pattern comprising a peak, in terms of 2θ, at about 8.0, about 16.5° and about 17.8°. In a further embodiment, crystalline form (A-I) of the compound of formula (A) has an X-ray powder diffraction pattern comprising a peak, in terms of 2θ, at about 8.0°, about 16.5°, about 17.8° and about 21.2°. In yet a further embodiment, crystalline form (A-I) of the compound of formula (A) has an X-ray powder diffraction pattern comprising a peak, in terms of 2θ, at about 8.0°, about 16.5°, about 17.8°, about 21.2° and about 24.5°. In yet a further embodiment, crystalline form (A-I) of the compound of formula (A) has an X-ray powder diffraction pattern substantially as shown in FIG. 1.

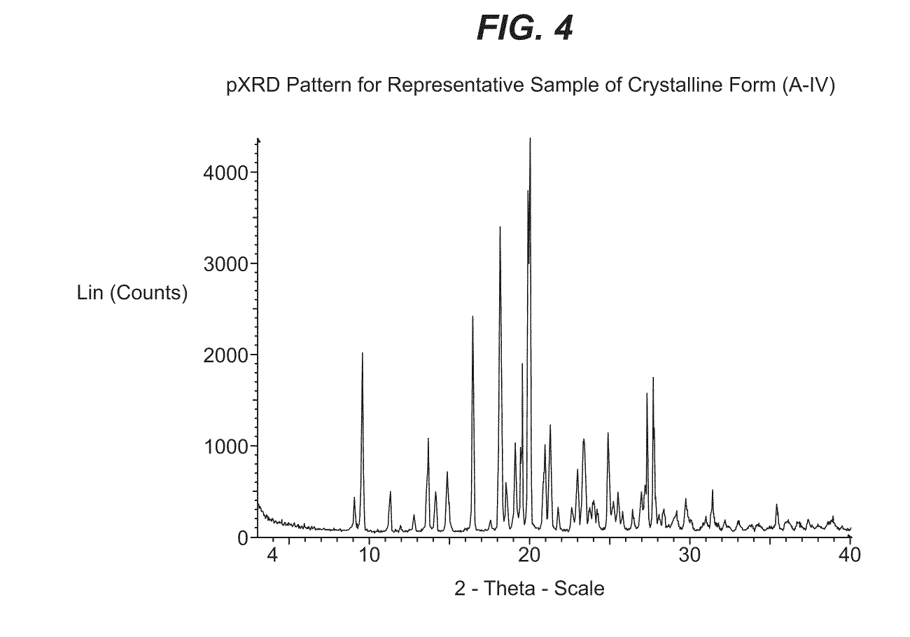

The powder XRD spectrum was measured for a representative sample of crystalline form (A-IV) of the compound of formula (A), as shown in FIG. 4. Crystalline form (A-IV) of the compound of formula (A) may be characterized by its powder X-ray diffraction pattern, which comprised the peaks as listed in Table 4, below.

TABLE 4
pXRD Peaks, Crystalline Form (A-IV)

| Pos. [°2Th.] | d-spacing [Å] | Rel. Int. [%] |
|---|---|---|
| 9.0 | 9.8 | 10 |
| 9.5 | 9.3 | 52 |
| 11.3 | 7.8 | 11 |
| 13.5 | 6.5 | 14 |
| 13.6 | 6.5 | 25 |
| 14.1 | 6.3 | 10 |
| 14.8 | 6.0 | 18 |
| 16.5 | 5.4 | 62 |
| 18.2 | 4.9 | 83 |
| 18.5 | 4.8 | 14 |
| 19.1 | 4.6 | 26 |
| 19.5 | 4.6 | 43 |
| 19.9 | 4.5 | 100 |
| 20.0 | 4.4 | 93 |
| 20.9 | 4.2 | 26 |
| 21.2 | 4.2 | 31 |
| 22.9 | 3.9 | 16 |
| 23.4 | 3.8 | 26 |
| 24.9 | 3.6 | 27 |
| 25.5 | 3.5 | 11 |
| 27.0 | 3.3 | 11 |
| 27.3 | 3.3 | 36 |
| 27.7 | 3.2 | 42 |
| 29.7 | 3.0 | 10 |
| 31.4 | 2.8 | 11 |

Preferably, crystalline form (A-IV) of the compound of formula (A) is characterized by its pXRD pattern which comprises peaks having a relative intensity greater than or equal to about 15%, more preferably, greater than or equal to about 25%, as listed in Table 5, below.

TABLE 5
pXRD Peaks, Crystalline Form (A-IV)

| Pos. [°2Th.] | d-spacing [Å] | Rel. Int. [%] |
|---|---|---|
| 9.5 | 9.3 | 52 |
| 13.6 | 6.5 | 25 |
| 16.5 | 5.4 | 62 |
| 18.2 | 4.9 | 83 |
| 19.1 | 4.6 | 26 |
| 19.5 | 4.6 | 43 |
| 19.9 | 4.5 | 100 |
| 20.0 | 4.4 | 93 |
| 20.9 | 4.2 | 26 |
| 21.2 | 4.2 | 31 |
| 23.4 | 3.8 | 26 |
| 24.9 | 3.6 | 27 |
| 27.3 | 3.3 | 36 |
| 27.7 | 3.2 | 42 |

In an embodiment, crystalline form (A-IV) of the compound of formula (A) is characterized by its pXRD pattern which comprises the peaks listed in Table 6, below.

TABLE 6
pXRD Peaks, Crystalline Form (A-IV)

| Pos. [°2Th.] | d-spacing [Å] |
|---|---|
| 9.5 | 9.3 |
| 16.5 | 5.4 |
| 18.2 | 4.9 |
| 19.9 | 4.5 |
| 23.4 | 3.8 |

In one embodiment, crystalline form (A-IV) of the compound of formula (A) has an X-ray powder diffraction pattern comprising a peak, in terms of 2θ, at about 19.9°. In another embodiment, crystalline form (A-IV) of the compound of formula (A) has an X-ray powder diffraction pattern comprising a peak, in terms of 2θ, at about 18.2° and about 19.9°. In yet another embodiment, crystalline form (A-IV) of the compound of formula (A) has an X-ray powder diffraction pattern comprising a peak, in terms of 2θ, at about 16.5°, about 18.2° and about 19.9°. In a further embodiment, crystalline form (A-IV) of the compound of formula (A) has an X-ray powder diffraction pattern comprising a peak, in terms of 2θ, at about 9.5°, about 16.5°, about 18.2° and about 19.9°. In yet a further embodiment, crystalline form (A-IV) of the compound of formula (A) has an X-ray powder diffraction pattern comprising a peak, in terms of 2θ, at about 9.5°, about 16.5°, about 18.2°, about 19.9° and about 23.4°. In yet a further embodiment, crystalline form (A-IV) of the compound of formula (A) has an X-ray powder diffraction pattern substantially as shown in FIG. 4.

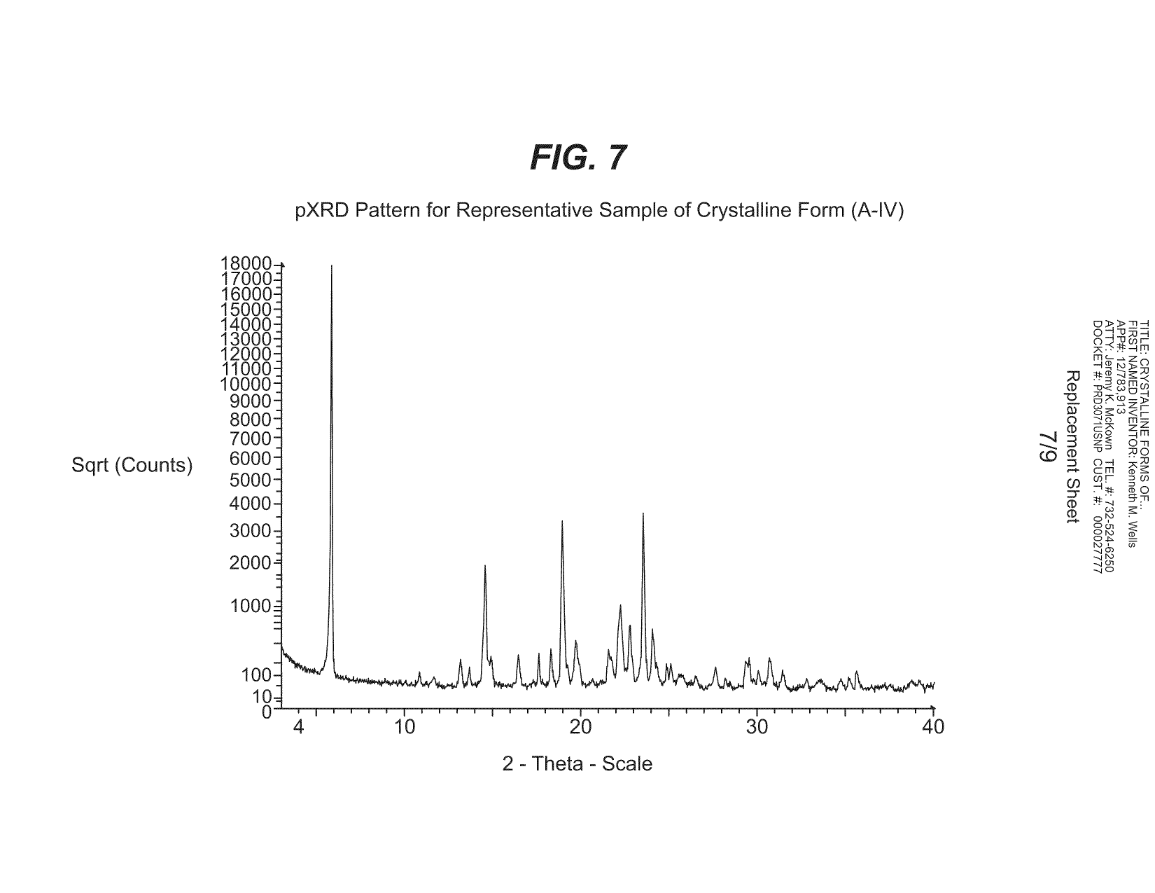

The powder XRD spectrum was measured for a representative sample of crystalline form (A-VI) of the compound of formula (A), as shown in FIG. 7. Crystalline form (A-VI) of the compound of formula (A) may be characterized by its powder X-ray diffraction pattern, which comprised the peaks as listed in Table 7, below.

TABLE 7
pXRD Peaks, Crystalline Form (A-VI)

| Pos. [°2Th.] | d-spacing [Å] | Rel. Int. [%] |
|---|---|---|
| 5.8 | 15.1 | 100 |
| 13.2 | 6.7 | 1 |
| 13.6 | 6.5 | 1 |
| 14.6 | 6.1 | 10 |
| 14.9 | 5.9 | 1 |
| 16.4 | 5.4 | 1 |
| 17.6 | 5.0 | 1 |
| 18.3 | 4.9 | 2 |
| 18.9 | 4.7 | 18 |
| 19.7 | 4.5 | 2 |
| 21.5 | 4.1 | 1 |
| 22.2 | 4.0 | 5 |
| 22.7 | 3.9 | 3 |
| 23.5 | 3.8 | 19 |
| 24.0 | 3.7 | 3 |
| 24.8 | 3.6 | 1 |
| 25.1 | 3.6 | 1 |

Preferably, crystalline form (A-VI) of the compound of formula (A) is characterized by its pXRD pattern which comprises peaks having a relative intensity greater than or equal to about 2%, as listed in Table 8, below.

TABLE 8
pXRD Peaks, Crystalline Form (A-VI)

| Pos. [°2Th.] | d-spacing [Å] | Rel. Int. [%] |
|---|---|---|
| 5.8 | 15.1 | 100 |
| 14.6 | 6.1 | 10 |
| 18.3 | 4.9 | 2 |
| 18.9 | 4.7 | 18 |
| 19.7 | 4.5 | 2 |
| 22.2 | 4.0 | 5 |
| 22.7 | 3.9 | 3 |
| 23.5 | 3.8 | 19 |
| 24.0 | 3.7 | 3 |

Preferably, crystalline form (A-VI) of the compound of formula (A) is characterized by its pXRD pattern which comprises peaks having a relative intensity greater than or equal to about 5%, as listed in Table 9, below.

TABLE 9
pXRD Peaks, Crystalline Form (A-VI)

| Pos. [°2Th.] | d-spacing [Å] | Rel. Int. [%] |
|---|---|---|
| 5.8 | 15.1 | 100 |
| 14.6 | 6.1 | 10 |
| 18.9 | 4.7 | 18 |
| 22.2 | 4.0 | 5 |
| 23.5 | 3.8 | 19 |

In an embodiment, crystalline form (A-VI) of the compound of formula (A) is characterized by its pXRD pattern which comprises the peaks listed in Table 10, below.

TABLE 10
pXRD Peaks, Crystalline Form (A-VI)

| Pos. [°2Th.] | d-spacing [Å] |
|---|---|
| 5.8 | 15.1 |
| 14.6 | 6.1 |
| 18.9 | 4.7 |
| 22.2 | 4.0 |
| 23.5 | 3.8 |

In one embodiment, crystalline form (A-VI) of the compound of formula (A) has an X-ray powder diffraction pattern comprising a peak, in terms of 2θ, at about 5.8°. In another embodiment, crystalline form (A-VI) of the compound of formula (A) has an X-ray powder diffraction pattern comprising a peak, in terms of 2θ, at about 5.8° and about 23.5°. In yet another embodiment, crystalline form (A-VI) of the compound of formula (A) has an X-ray powder diffraction pattern comprising a peak, in terms of 2θ, at about 5.8, about 18.9° and about 23.5°. In a further embodiment, crystalline form (A-VI) of the compound of formula (A) has an X-ray powder diffraction pattern comprising a peak, in terms of 2θ, at about 5.8°, about 14.6°, about 18.9° and about 23.5°. In yet a further embodiment, crystalline form (A-VI) of the compound of formula (A) has an X-ray powder diffraction pattern comprising a peak, in terms of 2θ, at about 5.8°, about 14.6°, about 18.9°, about 22.2° and about 23.5°. In yet a further embodiment, crystalline form (A-VI) of the compound of formula (A) has an X-ray powder diffraction pattern substantially as shown in FIG. 7.

### Differential Scanning Calorimetry (DSC)

The crystalline forms of the present invention were subjected to DSC analysis. A representative sample was tested using a TA Instruments DSC Q100 differential scanning calorimeter. The sample was analyzed as received in a crimped TA Instrument aluminum sample pan and was program heated from ambient to 250 ° C. at 10 ° C./min under nitrogen purge.

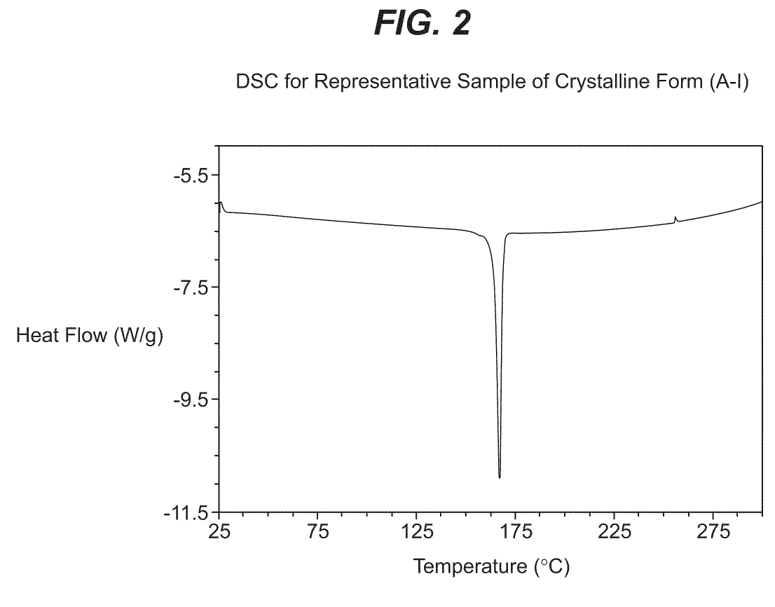

DSC was measured for a representative sample of crystalline form (A-I) of the compound of formula (A), as shown in FIG. 2. Crystalline form (A-I) of the compound of formula (A) exhibited an onset temperature of melting of 164.6° C., a peak temperature of melting of 166.8° C. and a heat of melting of 86.2 J/g.

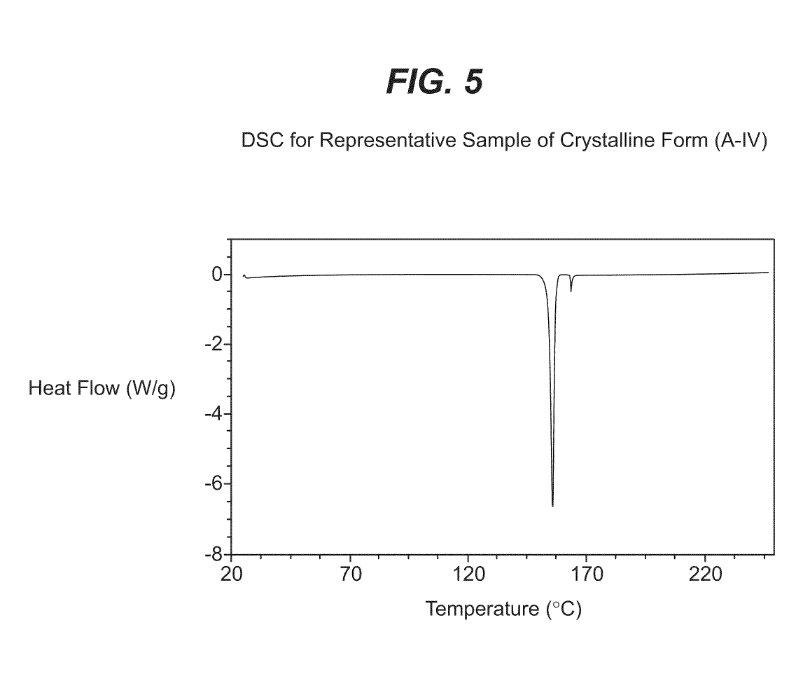

DSC was measured for a representative sample of crystalline form (A-IV) of the compound of formula (A), as shown in FIG. 5. Crystalline form (A-IV) of the compound of formula (A) exhibited an onset temperature of melting of 154.1° C., a peak temperature of melting of 155.6° C. and a heat of melting of 86.8 J/g.

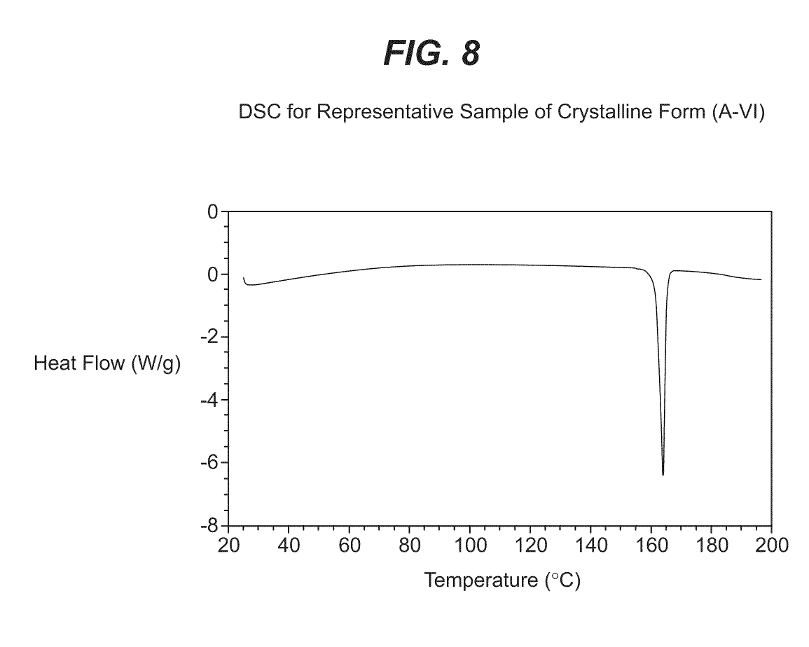

DSC was measured for a representative sample of crystalline form (A-VI) of the compound of formula (A), as shown in FIG. 8. Crystalline form (A-VI) of the compound of formula (A) exhibited an onset temperature of melting of 162.1° C., a peak temperature of melting of 164.0° C. and a heat of melting of 92.2 J/g.

### Thermogravimetric Analysis (TGA)

The crystalline forms of the present invention were subjected to DSC analysis. A representative sample was tested for weight loss using a TA Instruments TGA Q50 thermogravimetric calorimeter. The sample was analyzed as received and was program heated from ambient to 300° C. at 10 ° C./min under nitrogen purge.

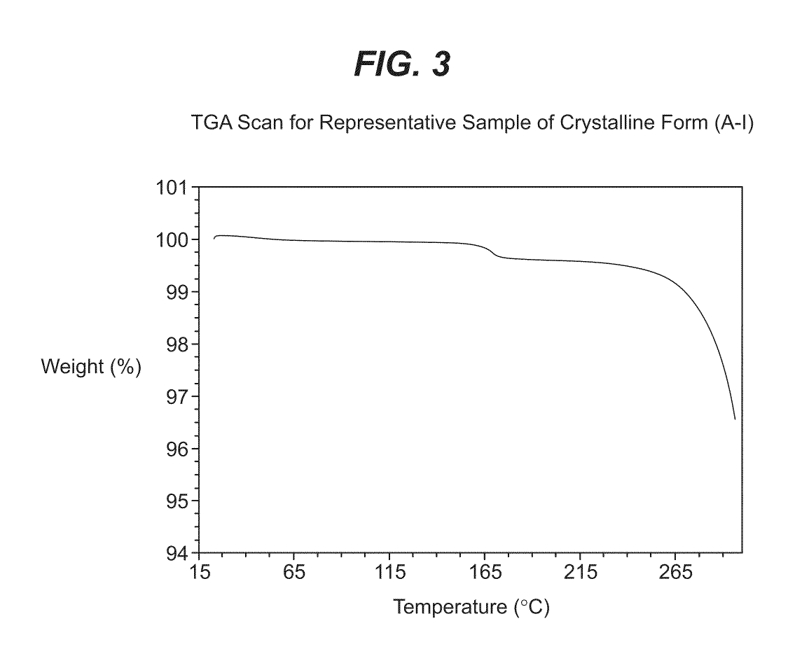

TGA was measured for a representative sample of crystalline form (A-I) of the compound of formula (A), as shown in FIG. 3. 0.3% weight loss was observed by TGA from ambient temperature up to 165° C. and including the melting of the sample. These results indicate that crystalline form (A-I) is an anhydrous form.

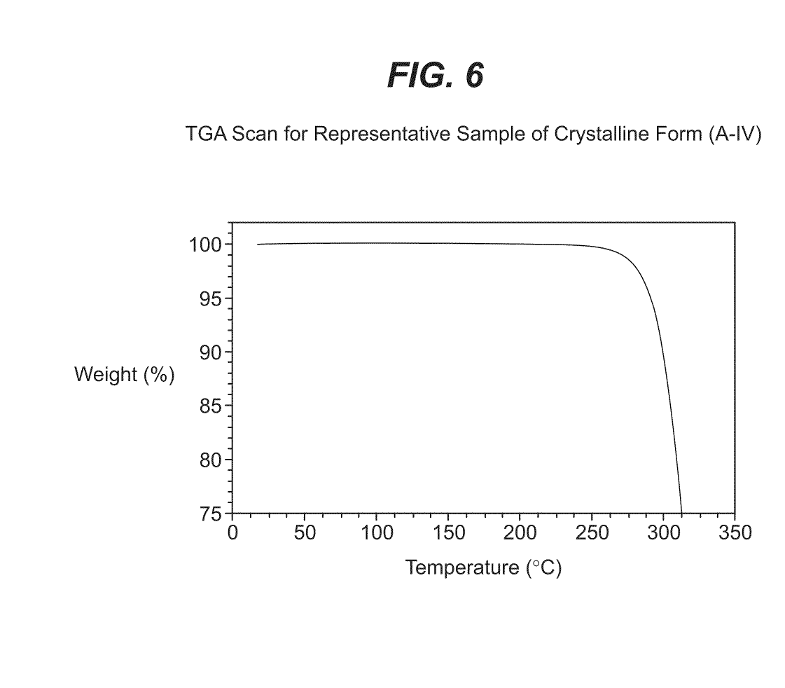

TGA was measured for a representative sample of crystalline form (A-IV) of the compound of formula (A), as shown in FIG. 6. 0.1% weight loss was observed by TGA from ambient temperature up to 165° C. and including the melting of the sample. These results indicate that crystalline form (A-IV) is an anhydrous form.

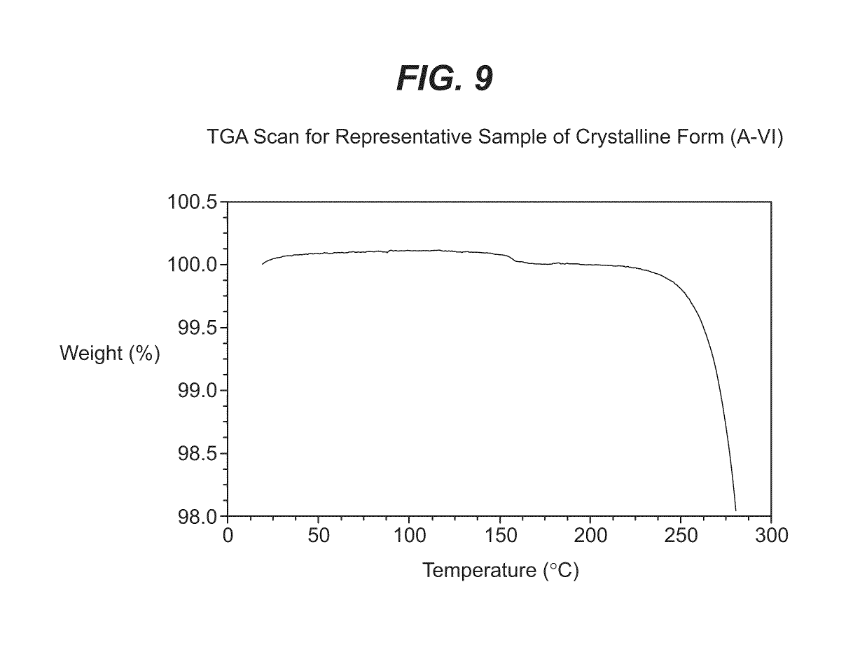

TGA was measured for a representative sample of crystalline form (A-VI) of the compound of formula (A), as shown in FIG. 9. 0.2% weight loss was observed by TGA from ambient temperature up to 165° C., and including the melting of the sample. These results indicate that crystalline form (A-VI) is an anhydrous form.

The present invention further comprises compositions containing any of the crystalline forms of the compound of formula (A) as described herein. In some embodiments, the compositions of the invention include at least about 50%, about 60%, about 70%, about 80%, about 90%, about 95%, about 96%, about 97%, about 98%, or about 99% by weight of the crystalline Form (A-I). In some embodiments, the compositions of the invention include at least about 50%, about 60%, about 70%, about 80%, about 90%, about 95%, about 96%, about 97%, about 98%, or about 99% by weight of the crystalline Form (A-IV). In some embodiments, the compositions of the invention include at least about 50%, about 60%, about 70%, about 80%, about 90%, about 95%, about 96%, about 97%, about 98%, or about 99% by weight of the crystalline Form (A-VI). In some embodiments, the compositions of the invention contain a mixture of two or more of crystalline Form (A-I), crystalline Form (A-IV) and/or crystalline Form (A-VI). In some embodiments, compositions of the invention include one or more of crystalline Form (A-I), crystalline Form (A-IV) and/or crystalline Form (A-VI), and a pharmaceutically acceptable carrier.

The present invention further comprises pharmaceutical compositions containing any of the crystalline forms of the compound of formula (A) described herein with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenterals, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.1 to about 1000 mg or any range therein, and may be given at a dosage of from about 0.01 to about 300 mg/kg/day, or any range therein, preferably from about 0.1 to about 50 mg/kg/day, or any range therein. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from about 0.1 to about 1000 mg, or any range or amount therein, of the active ingredient (any of the crystalline forms of the compound of formula (A) as described herein). The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The methods of treatment described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any range or amount therein; preferably about 1.0 to about 500 mg, or any range or amount therein, of any of the crystalline forms of the compound of formula (A) as described herein, and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixers, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, any of the crystalline forms of the compound of formula (A) as described herein, as the active ingredient, is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be found in The Handbook of Pharmaceutical Excipients, published by the American Pharmaceutical Association and the Pharmaceutical Society of Great Britain.

Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of this invention may be administered in any of the foregoing compositions and according to dosage regimens established in the art whenever treatment of anxiety and related disorders; bipolar depression and mania; depression; epilepsy and related disorders; epileptogenesis; glucose related disorders; lipid related disorders; migraine; obesity; pain; substance abuse or neuroprotection is required.

The daily dosage of the products may be varied over a wide range from about 0.1 to about 7,000 mg per adult human per day, or any range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250, 500 and 1000 milligrams of the active ingredient for the symptomatic adjustment of the dosage to the patient to be treated. An effective amount of the drug is ordinarily supplied at a dosage level of from about 0.01 mg/kg to about 100 mg/kg of body weight per day, or any range therein. Preferably, the range is from about 0.1 to about 100 mg/kg of body weight per day, or any range therein. More preferably, from about 0.5 to about 50 mg/kg of body weight per day, or any range therein. The compounds may be administered on a regimen of 1 to 4 times per day.

Optimal dosages to be administered may be readily determined by those skilled in the art, and will vary with the particular compound used, the mode of administration, the strength of the preparation, the mode of administration, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder. One skilled in the art will further recognize that human clinical trials including first-in-human, dose ranging and efficacy trials, in healthy patients and/or those suffering from a given disorder, may be completed according to methods well known in the clinical and medical arts.

The following Examples are set forth to aid in the understanding of the invention, and are not intended and should not be construed to limit in any way the invention set forth in the claims which follow thereafter.

### Example 1

### Crystalline Form (A-I) of 4-[6-(6-Methanesulfonyl-2-methyl-pyridin-3-ylamino)-5-methoxy-pyrimidin-4-yloxy]-piperidine-1-carboxylic acid isopropyl ester

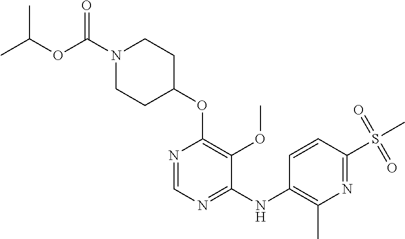

A 5-L 3-neck flask equipped with an overhead mechanical stirrer, N₂ inlet/outlet adapter, reflux condenser, and thermocouple was charged with 1,4-dioxane (715 mL) and degassed for 10 min. 1,1′-Bis(di-tert-butylphosphino)ferrocene (22.9 g, 0.046 mol), and Pd(OAc)₂ (5.1 g, 0.023 mol) was added at room temperature and the resulting mixture was degassed and purged with N₂ three times. The resulting heterogeneous solution was heated to 75° C. and stirred for 30 min. The resulting mixture was then cooled to room temperature and sodium t-butoxide (62.5 g, 0.65 mol) was added all at once. The catalyst solution was degassed and purged with N₂ three times. 4-(6-chloro-5-methoxy-pyrimidin-4-yloxy)-piperidine-1-carboxylic acid isopropyl ester (143.0 g, 0.43 mol) and 2-methyl-6-(methylsulfonyl)-3-pyridinamine (80.8 g, 0.43 mol) were then added together with 1,4-dioxane (715 mL) to the catalyst solution via addition funnel over 10 min. The resulting mixture was heated to 50° C. for 18 h. The resulting mixture was cooled to room temperature, poured into ethyl acetate (2L), and washed with 1N HCl (2×500 mL). The organic layer was then washed with brine (500 mL), dried over MgSO₄, filtered, and concentrated to dryness to yield the compound of formula (A) as a residue.

The residue was purified using the Isco LC prep to yield the compound of formula (A) as a yellow solid (81% isolated yield, 99% HPLC purity).

The chromatographed yellow solid was re-crystallized from ethyl acetate (350 mL) and heptane (450 mL) as follows. The slurry was heated to 75° C. for 30 min. The resulting slurry was cooled to room temperature over 30 min and then aged 30 min. The resulting thick slurry was filtered, rinsed with heptane (3×25 mL), and placed into a vacuum oven at 40° C. for 36 h to yield crystalline form (A-I) of the compound of formula (A) as a solid.

Examples 2-4 recite recipes/procedures for the synthesis of the titled compound(s) and/or crystalline form(s). Several batches of the said compounds were prepared according to the recipes/procedures, with results as discussed at the end of each example.

### Example 2

### 4-(6-Chloro-5-methoxy-pyrimidin-4-yloxy)-piperidine-1-carboxylic acid isopropyl ester

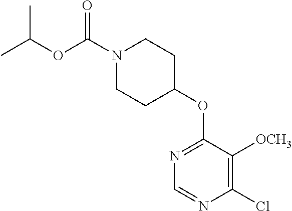

### Example 2a

### NaH Process

A 500 ml, 4-necked flask equipped with thermometer, mechanical stirrer and condenser with gas inlet was purged with N₂ and charged with NaH (4.4 g; 0.11 mol) and N,N-dimethylformamide (50 ml). In a separate flask were dissolved 4-hydroxy-piperidine-1-carboxylic acid isopropyl ester (18.7 g; 0.1 mol) and 4,6-dichloro-5-methoxy-pyrimidine (17.9 g; 0.1 mol) in DMF (50 ml; 0.5 L/mol). The prepared solution was then added dropwise to the above-mentioned NaH/DMF suspension while maintaining the temperature between −10 and −5° C. The resulting mixture is then stirred for one hour, then allowed to warm up to room temperature and stirred for 17 hours. Water (300 ml; 3 L/mol) was added dropwise while maintaining the temperature between 15-30° C. by cooling with tap water. Heptane (125 ml; 1.25 L/mol) was added and the resulting mixture was heated up to 55° C. The aqueous layer was discarded; the organic layer was cooled down to 20° C. and stirred for another 3-20 h. The resulting precipitate was filtered and dried in vacuum@50° C. for 20 h to yield the title compound as a residue.

### Example 2b

### KO-t-Bu Process

A 1 L, 4-necked flask equipped with thermometer, mechanical stirrer and condenser with gas inlet was purged with N₂ and charged with 4-hydroxy-piperidine-1-carboxylic acid isopropyl ester (18.7 g; 0.1 mol), toluene (200 ml; 2 L/mol) and 4,6-dichloro-5-methoxy-pyrimidine (17.8 g; 1 eq.). The resulting mixture was cooled down to 10° C. A solution of 1N KO-t-Bu in THF (110 ml; 1.1 eq.) was then added dropwise over 10 min. After 30 min, the resulting mixture was allowed to heat to room temperature. After 1 h at room temperature, the resulting mixture was quenched with water (200 ml; 2 L/mol) and stirred for 10 min. The aqueous layer was discarded and the organic layer was evaporated to dryness under vacuum. Heptane (60 ml; 0.6 L/mol) was added and the resulting mixture was heated up to 55-60° C. (colorless transparent solution), then cooled to 45° C., seeded with polymorph form IV and further cooled down to room temperature over 2 h. The resulting crystallized product (heavy crystals) was filtered off and washed with heptane (15 ml; 0.15 L/mol), then dried (45° C., vac., 2 h) to yield the title compound.

### Example 3

### Crystalline Form (A-IV) of 4-[6-(6-Methanesulfonyl-2-methyl-pyridin-3-ylamino)-5-methoxy-pyrimidin-4-yloxy]-piperidine-1-carboxylic acid isopropyl ester

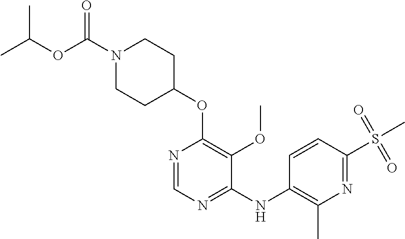

### Example 3a

### Step A: Catalyst Preparation

A 250 ml, 4-necked flask equipped with thermometer, mechanical stirrer and condenser with gas inlet is purged with argon and charged with toluene (120 ml; 1 L/mol). The toluene was degassed by bubbling with argon for 15 min. Bis(2-diphenylphosphinophenyl)ether (2.59 g; 4 mol %) was dissolved under a stream of argon. Palladium(II)acetate (0.54 g; 2 mol %) was added in one portion; with the orange powder observed to go into solution. After a few minutes a solid appeared and a yellow suspension was observed.

### Step B: Coupling Reaction

A 250 ml, 4-necked flask equipped with thermometer, mechanical stirrer and condenser with gas inlet was purged with Argon and then charged with toluene (360 ml, 3 L/mol). The toluene was degassed by bubbling with argon for 15 minutes. 4-(6-Chloro-5-methoxy-pyrimidin-4-yloxy)-piperidine-1-carboxylic acid isopropyl ester (39.6 g; 0.12 mol), 6-methanesulfonyl-2-methyl-pyridin-3-ylamine (22.3 g; 0.12 mol) and NaO-t-Bu (17.3 g; 1.5 eq.) were added at room temperature. The resulting suspension was heated to 60° C. while being degassed by bubbling with Argon. The catalyst suspension prepared as in STEP A above was added in one portion to the reaction mixture (the catalyst suspension is fluid enough to be added via an addition funnel). After 30 minutes, the Ar-stream was stopped and switched to a N₂ stream. The resulting mixture was stirred over 16-18 h at 60° C.

Water (120 ml; 1 L/mol) was then added to the hot reaction mixture while maintaining the temperature at 60° C. The solid was observed to dissolve. A brown organic layer was obtained after discarding the aqueous one together with the black interface. Aqueous 1M HCl (120 ml; 1.2 eq.) was added and the color of the mixture was observed to go from brown to light brown. The resulting mixture was then heated to 60° C. The same extraction procedure was carried out a second time (aqueous layer discarded, organic layer heated to 60° C.).

To the resulting mixture was added water (120 ml, 1.2 L/mol) and the aqueous layer was discarded. To the organic layer was then added sodium sulfate (9.0 g; 75 g/mol), Silica Gel Thiol 3 (11.4 g; 95 g/mol) and NORIT® A SUPRA (2.4 g; 20 g/mol) and the resulting mixture stirred for 30 min at 60° C.; then filtered over DICALITE. The filter cake was rinsed with toluene (12 ml; 0.1 L/mol) to yield the title compound as a solid.

The above described procedure was run four times, the first three batches were prepared using the NaH process and the fourth batch was prepared using the KO-t-Bu process. For each batch (X1, X2, X3 and X4, respectively), the product isolated (prior to the crystallization as described below) was determined to be crystalline polymorph form (A-IV), as measured by powder XRD.

### Example 3b

### Crystallization

Toluene was stripped off (+/−15% of total toluene) from the filtercake prepared as in Example 3a above, and then isopropanol (640 ml; 5.33 L/mol) was added. To the resulting solution was then added Silica Gel Thiol 3 (11.4 g; 95 g/mol) and NORIT® A SUPRA (2.4 g; 20 g/mol) and the resulting mixture stirred for 30 min at 60° C., then filtered over DICALITE. The filtercake was rinsed with isopropanol (12 ml; 0.1 L/mol). The remaining toluene was distilled with the isopropanol (azeotrope@81° C.). The residual solution was left to cool down slowly to room temperature. The resulting mixture was filtered on a filter paper and the solid rinsed with isopropanol (24 ml; 0.2 L/mol). The solid was dried for 18 h in an oven under vacuum at 50° C. with a stream of N₂ to yield the title compound.

The crystallization process described above was applied to the product isolated from batches X1 and X2, as described in Example 3a above. The isolated product determined by powder XRD to be crystalline form (A-IV).

### Example 3c

In a separate example, crystalline form (A-IV) was prepared by dissolving crystalline form (A-I) at about 60 mg/mL in isopropanol, heating the resulting mixture at a rate of about 1°/min to reflux temperature; and then cooling at a rate of about 0.25° C./min, to yield a solid precipitate. The precipitate was observed to about at a temperature of less than about 55° C., and was collected by filtration.

### Example 3d

In yet another example, crystalline form (A-IV) was also prepared by dissolving crystalline form (A-I) at about 90 mg/mL in ethanol, heating the resulting mixture at a rate of about 1° C./min to reflux; and then cooling at a rate of about 0.25° C./min, to yield a solid precipitate.

### Example 4

### Crystalline Form (A-VI) of 4-[6-(6-Methanesulfonyl-2-methyl-pyridin-3-ylamino)-5-methoxy-pyrimidin-4-yloxy]-piperidine-1-carboxylic acid isopropyl ester

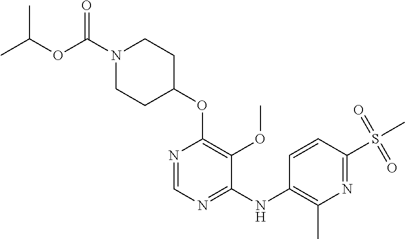

### Example 4a

Toluene was stripped off (+/−15% of total toluene) from the filtercake prepared as in Example 3a above, and then isopropanol (640 ml; 5.33 L/mol) was added. To the resulting solution was then added Silica Gel Thiol 3 (11.4 g; 95 g/mol) and NORIT® A SUPRA (2.4 g; 20 g/mol) and the resulting mixture stirred for 30 min at 60° C., then filtered over DICALITE. The filtercake was rinsed with isopropanol (12 ml; 0.1 L/mol). The remaining toluene was distilled with the isopropanol (azeotrope at 81° C.). The residual solution was left to cool down slowly to room temperature. The resulting mixture was filtered on a filter paper and the solid rinsed with isopropanol (24 ml; 0.2 L/mol). The solid was dried for 18 h in an oven under vacuum at 50° C. with a stream of N₂ to yield the title compound.

The crystallization process described above (which is identical to the one described in Example 3b above) was applied to the product isolated from batches X3 and X4 as described in Example 3a above. The isolated product for these batches was determined (by powder XRD) to be crystalline form (A-VI).

### Example 4b

A 250 ml, 4-necked flask equipped with thermometer, mechanical stirrer and condenser with a gas inlet was purged with N₂ and charged with ethanol (80 ml; 4.8 L/mol), 4-[6-(6-methanesulfonyl-2-methyl-pyridin-3-ylamino)-5-methoxy-pyrimidin-4-ylmethyl]-piperidine-1-carboxylic acid isopropyl ester (8.0 g; 0.0167 mol) and Silica Gel Thiol 3 (2.5 g; 150 g/mol). The resulting suspension was heated to reflux (75-76° C.), stirred at this temperature for 45 min, then cooled to 70-72° C. The resulting warm suspension was filtered over DICALITE to remove the silica gel and the flask was rinsed with ethanol (20 ml; 1.2 L/mol).

The filtrate was then reheated to reflux and stirred for 15 min; then to 57° C. over 1 h. The resulting solution was seeded, stirred at 50-55° C. for 30 minutes, cooled down further to 20° C. over 2 h then further cooled to 5° C. over 1 h. The resulting mixture was stirred at 5° C. for 1 h, filtered and dried (40° C., vacuum, N₂, 18 h) to yield a solid, which was determined (by powder XRD) to be crystalline form (A-VI).

Multiple batches of the process described in Example 4b were run, seeding with either crystalline form (A-IV) or crystalline form (A-VI). Regardless of the crystalline form used for the seeding, the product obtained was determined by powder XRD to be crystalline form (A-VI).

### Example 5

### Oral Formulation—Prophetic Example

As a specific embodiment of an oral composition, 100 mg of crystalline form (A-I), prepared as in Example 1, is formulated with sufficient finely divided lactose to provide a total amount of 580 to 590 mg to fill a size O hard gel capsule.

### Example 6

### Oral Formulation—Prophetic Example

As a specific embodiment of an oral composition, 100 mg of crystalline form (A-IV), prepared as in Example 3, is formulated with sufficient finely divided lactose to provide a total amount of 580 to 590 mg to fill a size O hard gel capsule.

### Example 7

### Oral Formulation—Prophetic Example

As a specific embodiment of an oral composition, 100 mg of crystalline form (A-VI), prepared as in Example 4, is formulated with sufficient finely divided lactose to provide a total amount of 580 to 590 mg to fill a size 0 hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

We claim:

1. A crystalline form of a compound of formula (A):

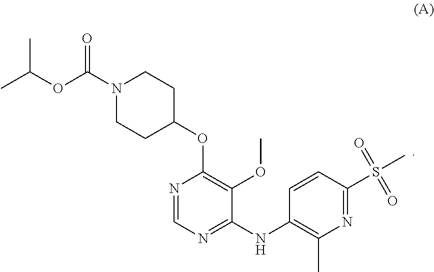

2. Crystalline form (A-IV) of a compound of formula (A) 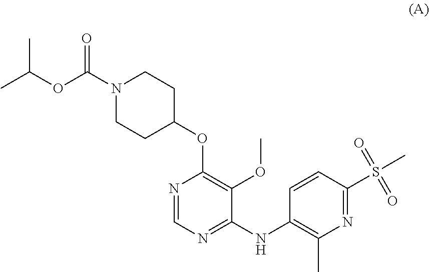
having an X-ray powder diffraction pattern comprising a peak, in terms of 2θ, at about 19.9°.

3. Crystalline form (A-IV) as in claim 2, having an X-ray powder diffraction pattern comprising a peak, in terms of 2θ, at about 18.2° and about 19.9°.

4. Crystalline form (A-IV) as in claim 2,having an X-ray powder diffraction pattern comprising a peak, in terms of 2θ, at about 16.5°, about 18.2°, and about 19.9°.

5. Crystalline form (A-IV) as in claim 2, having an X-ray powder diffraction pattern comprising a peak, in terms of 2θ, at about 9.5°, about 16.5°, about 18.2°, and about 19.9°.

6. Crystalline form (A-IV) as in claim 2, having an X-ray powder diffraction pattern comprising a peak, in terms of 2θ, at about 9.5°, about 16.5°, about 18.2°, about 19.9°, and about 23.4°.

7. Crystalline form (A-IV) as in claim 2, having an X-ray powder diffraction pattern substantially as shown in FIG. 4.

8. A composition comprising said crystalline form (A-IV) as in claim 2.

9. A composition as in claim 8, wherein said crystalline form (A-IV) constitutes at least about 50% by weight of said composition.

10. A composition as in claim 8, wherein said crystalline form (A-IV) constitutes at least about 90% by weight of said composition.

11. A composition as in claim 8, wherein said crystalline form (A-IV) constitutes at least about 99% by weight of said composition.

12. A pharmaceutical composition comprising a pharmaceutically acceptable carrier and crystalline form (A-IV) as in claim 2.

13. A pharmaceutical composition made by mixing crystalline form (A-IV) as in claim 2 and a pharmaceutically acceptable carrier.

14. A process for making a pharmaceutical composition comprising mixing crystalline form (A-IV) as in claim 2 and a pharmaceutically acceptable carrier.

15. Crystalline form (A-IV) as in claim 2 for use in a method of treatment of the human or animal body by therapy.

16. Crystalline form (A-IV) as in claim 2 for use in a method of treatment of a metabolic related disorder.

17. Crystalline form (A-IV) as in claim 2 for use in a method of treatment of a metabolic related disorder selected from the group consisting of Type I diabetes, Type II diabetes, inadequate glucose tolerance, insulin resistance, hyperglycemia, hyperlipidemia, hypertriglyceridemia, hypercholesterolemia, dyslipidemia, and Syndrome X.

18. Crystalline form (A-IV) as in claim 2 for use in a method of treatment of Type II diabetes.

19. Crystalline form (A-IV) as in claim 2 for use in a method of (a) decreasing food intake, (b) inducing satiety, (c) controlling weight gain, or (d) decreasing weight gain, in a subject in need thereof.

20. A method of treating a metabolic related disorder, comprising administering to a subject in need thereof a therapeutically effective amount of crystalline form (A-IV) as in claim 2.

21. The method as in claim 20, wherein the metabolic related disorder is selected from the group consisting of Type I diabetes, Type II diabetes, inadequate glucose tolerance, insulin resistance, hyperglycemia, hyperlipidemia, hypertriglyceridemia, hypercholesterolemia, dyslipidemia, and Syndrome X.

22. The method as in claim 20, wherein the metabolic related disorder is Type II diabetes.

23. A method of decreasing food intake, inducing satiety, controlling weight gain or decreasing weight gain comprising administering to a subject in need thereof, a therapeutically effective amount of crystalline form (A-IV) as in claim 2.

24. Use of crystalline form (A-IV) as in claim 2 for the preparation of a medicament for treating a metabolic related disorder in a subject in need thereof.

25. Use of crystalline form (A-IV) as in claim 2 for the preparation of a medicament for treating: (a) Type I diabetes, (b) Type II diabetes, (c) inadequate glucose tolerance, (d) insulin resistance, (e) hyperglycemia, (f) hyperlipidemia, (g) hypertriglyceridemia, (h) hypercholesterolemia, (i) dyslipidemia, or (j) Syndrome X, in a subject in need thereof.

26. Use of crystalline form (A-IV) as in claim 2 for the preparation of a medicament for treating Type II diabetes in a subject in need thereof.

27. Use of crystalline form (A-IV) as in claim 2 for the preparation of a medicament for (a) decreasing food intake, (b) inducing satiety, (c) controlling weight gain, or (d) decreasing weight gain, in a subject in need thereof.

28. Crystalline form (A-VI) of a compound of formula (A) 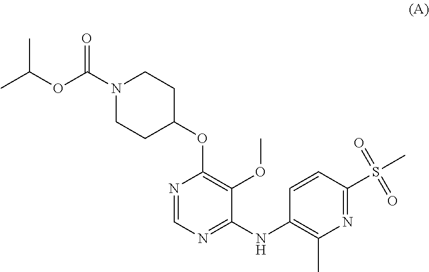
having an X-ray powder diffraction pattern comprising a peak, in terms of 2θ, at about 5.8°.

29. Crystalline form (A-VI) as in claim 28, having an X-ray powder diffraction pattern comprising a peak, in terms of 2θ, at about at about 5.8° and about 23.5°.

30. Crystalline form (A-VI) as in claim 28, having an X-ray powder diffraction pattern comprising a peak, in terms of 2θ, at about at about 5.8°, about 18.9°, and about 23.5°.

31. Crystalline form (A-VI) as in claim 28, having an X-ray powder diffraction pattern comprising a peak, in terms of 2θ, at about at about 5.8°, about 14.6°, about 18.9° and about 23.5°.

32. Crystalline form (A-VI) as in claim 28, having an X-ray powder diffraction pattern comprising a peak, in terms of 2θ, at about 5.8°, about 14.6°, about 18.9°, about 22.2° and about 23.5°.

33. Crystalline form (A-VI) as in claim 28, having an X-ray powder diffraction pattern substantially as shown in FIG. 7.

34. A composition comprising said crystalline form (A-VI) as in claim 28.

35. A composition as in claim 34, wherein said crystalline form (A-VI) constitutes at least about 50% by weight of said composition.

36. A composition as in claim 34, wherein said crystalline form (A-VI) constitutes at least about 90% by weight of said composition.

37. A composition as in claim 34, wherein said crystalline form (A-VI) constitutes at least about 99% by weight of said composition.

38. A pharmaceutical composition comprising a pharmaceutically acceptable carrier and crystalline form (A-VI) as in claim 28.

39. A pharmaceutical composition made by mixing crystalline form (A-VI) as in claim 28 and a pharmaceutically acceptable carrier.

40. A process for making a pharmaceutical composition comprising mixing crystalline form (A-VI) as in claim 28 and a pharmaceutically acceptable carrier.

41. Crystalline form (A-VI) as in claim 28 for use in a method of treatment of the human or animal body by therapy.

42. Crystalline form (A-VI) as in claim 28 for use in a method of treatment of a metabolic related disorder.

43. Crystalline form (A-VI) as in claim 28 for use in a method of treatment of a metabolic related disorder selected from the group consisting of Type I diabetes, Type II diabetes, inadequate glucose tolerance, insulin resistance, hyperglycemia, hyperlipidemia, hypertriglyceridemia, hypercholesterolemia, dyslipidemia, and Syndrome X.

44. Crystalline form (A-VI) as in claim 28 for use in a method of treatment of Type II diabetes.

45. Crystalline form (A-VI) as in claim 28 for use in a method of (a) decreasing food intake, (b) inducing satiety, (c) controlling weight gain, or (d) decreasing weight gain, in a subject in need thereof.

46. A method of treating a metabolic related disorder, comprising administering to a subject in need thereof a therapeutically effective amount of crystalline form (A-VI) as in claim 28.

47. The method as in claim 46, wherein the metabolic related disorder is selected from the group consisting of Type I diabetes, Type II diabetes, inadequate glucose tolerance, insulin resistance, hyperglycemia, hyperlipidemia, hypertriglyceridemia, hypercholesterolemia, dyslipidemia and Syndrome X.

48. The method as in claim 46, wherein the metabolic related disorder is Type II diabetes.

49. A method of decreasing food intake, inducing satiety, controlling weight gain or decreasing weight gain comprising administering to a subject in need thereof, a therapeutically effective amount of crystalline form (A-VI) as in claim 28.

50. Use of crystalline form (A-VI) as in claim 28 for the preparation of a medicament for treating a metabolic related disorder in a subject in need thereof.

51. Use of crystalline form (A-VI) as in claim 28 for the preparation of a medicament for treating: (a) Type I diabetes, (b) Type II diabetes, (c) inadequate glucose tolerance, (d) insulin resistance, (e) hyperglycemia, (f) hyperlipidemia, (g) hypertriglyceridemia, (h) hypercholesterolemia, (i) dyslipidemia, or (j) Syndrome X, in a subject in need thereof.

52. Use of crystalline form (A-VI) as in claim 28 for the preparation of a medicament for treating Type II diabetes in a subject in need thereof.

53. Use of crystalline form (A-VI) as in claim 28 for the preparation of a medicament for (a) decreasing food intake, (b) inducing satiety, (c) controlling weight gain, or (d) decreasing weight gain, in a subject in need thereof.

54. Crystalline form (A-I) of a compound of formula (A) 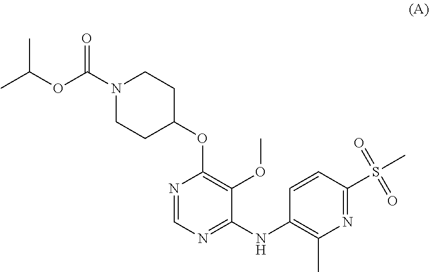
having an X-ray powder diffraction pattern comprising a peak, in terms of 2θ, at about at about 5.8°.

55. Crystalline form (A-I) as in claim 54, having an X-ray powder diffraction pattern comprising a peak, in terms of 2θ, at about 8.0° and about 16.4°.

56. Crystalline form (A-I) as in claim 54, having an X-ray powder diffraction pattern comprising a peak, in terms of 2θ, at about 8.0, about 16.4° and about 21.2°.

57. Crystalline form (A-I) as in claim 54, having an X-ray powder diffraction pattern comprising a peak, in terms of 2θ, at about 8.0, about 16.4°, about 17.7° and about 21.2°.

58. Crystalline form (A-I) as in claim 54, having an X-ray powder diffraction pattern comprising a peak, in terms of 2θ, at about 8.0°, about 16.4°, about 17.7°, about 21.2° and about 24.5°.

59. Crystalline form (A-I) as in claim 54, having an X-ray powder diffraction pattern substantially as shown in FIG. 1.

60. A composition comprising said crystalline form (A-I) as in claim 54.

61. A composition as in claim 60, wherein said crystalline form (A-I) constitutes at least about 50% by weight of said composition.

62. A composition as in claim 60, wherein said crystalline form (A-I) constitutes at least about 90% by weight of said composition.

63. A composition as in claim 60, wherein said crystalline form (A-I) constitutes at least about 99% by weight of said composition.

64. A pharmaceutical composition comprising a pharmaceutically acceptable carrier and crystalline form (A-I) as in claim 54.

65. A pharmaceutical composition made by mixing crystalline form (A-I) as in claim 54 and a pharmaceutically acceptable carrier.

66. A process for making a pharmaceutical composition comprising mixing crystalline form (A-I) as in claim 54 and a pharmaceutically acceptable carrier.

67. Crystalline form (A-I) as in claim 54 for use in a method of treatment of the human or animal body by therapy.

68. Crystalline form (A-I) as in claim 54 for use in a method of treatment of a metabolic related disorder.

69. Crystalline form (A-I) as in claim 54 for use in a method of treatment of a metabolic related disorder selected from the group consisting of Type I diabetes, Type II diabetes, inadequate glucose tolerance, insulin resistance, hyperglycemia, hyperlipidemia, hypertriglyceridemia, hypercholesterolemia, dyslipidemia, and Syndrome X.

70. Crystalline form (A-I) as in claim 54 for use in a method of treatment of Type II diabetes.

71. Crystalline form (A-I) as in claim 54 for use in a method of (a) decreasing food intake, (b) inducing satiety, (c) controlling weight gain, or (d) decreasing weight gain, in a subject in need thereof.

72. A method of treating a metabolic related disorder, comprising administering to a subject in need thereof a therapeutically effective amount of crystalline form (A-I) as in claim 54.

73. The method as in claim 72, wherein the metabolic related disorder is selected from the group consisting of Type I diabetes, Type II diabetes, inadequate glucose tolerance, insulin resistance, hyperglycemia, hyperlipidemia, hypertriglyceridemia, hypercholesterolemia, dyslipidemia and Syndrome X.

74. The method as in claim 72, wherein the metabolic related disorder is Type II diabetes.

75. A method of decreasing food intake, inducing satiety, controlling weight gain or decreasing weight gain comprising administering to a subject in need thereof, a therapeutically effective amount of crystalline form (A-I) as in claim 54.

76. Use of crystalline form (A-I) as in claim 54 for the preparation of a medicament for treating a metabolic related disorder in a subject in need thereof.

77. Use of crystalline form (A-I) as in claim 54 for the preparation of a medicament for treating: (a) Type I diabetes, (b) Type II diabetes, (c) inadequate glucose tolerance, (d) insulin resistance, (e) hyperglycemia, (f) hyperlipidemia, (g) hypertriglyceridemia, (h) hypercholesterolemia, (i) dyslipidemia, or (j) Syndrome X, in a subject in need thereof.

78. Use of crystalline form (A-I) as in claim 54 for the preparation of a medicament for treating Type II diabetes in a subject in need thereof.

79. Use of crystalline form (A-I) as in claim 54 for the preparation of a medicament for (a) decreasing food intake, (b) inducing satiety, (c) controlling weight gain, or (d) decreasing weight gain, in a subject in need thereof.
